(19) United States
(12) Patent Application Publication
(10) Pub. No.: US 2006/0073521 A1
(43) Pub. Date: Apr. 6, 2006

(54) Method for forming a film by spin coating

(75) Inventors: Yukou Saito, Kanagawa (JP); Hirohiko Tsuzuki, Kanagawa (JP)

Correspondence Address: SUGHRUE MION, PLLC, 2100 PENNSYLVANIA AVENUE, N.W., SUITE 800, WASHINGTON, DC, 20037, US

(73) Assignee: FUJI PHOTO FILM CO., LTD.

(21) Appl. No.: 11/238,013

(22) Filed: Sep. 29, 2005

(30) Foreign Application Priority Data
Sep. 30, 2004 (JP) 285861/2004

(51) Int. Cl. G01N 1/28; G01N 33/53; C12M 1/34; B05D 3/00

(52) U.S. Cl. 435/7.1; 435/287.2; 427/2.11

(57) ABSTRACT

It is an object of the present invention to provide a method for producing a solid substrate used for sensors having a film with a small film thickness distribution, and a solid surface used for sensors having a film with a small film thickness distribution. The present invention provides a method for spin coating wherein the substrate is rotated in a state where the substrate surface to be coated is inclined against the rotation surface during coating.

### TECHNICAL FIELD

The present invention relates to a method for forming a thin film with a small film thickness distribution, and a solid substrate used for sensors having a film with a small film thickness distribution. More specifically, the present invention relates to a solid substrate used for sensors, the surface of which is coated with a thin polymer film, and a production method thereof.

### BACKGROUND ART

Recently, a large number of measurements using intermolecular interactions such as immune responses are being carried out in clinical tests, etc. However, since conventional methods require complicated operations or labeling substances, several techniques are used that are capable of detecting the change in the binding amount of a test substance with high sensitivity without using such labeling substances. Examples of such a technique may include a surface plasmon resonance (SPR) measurement technique, a quartz crystal microbalance (QCM) measurement technique, and a measurement technique of using functional surfaces ranging from gold colloid particles to ultra-fine particles. The SPR measurement technique is a method of measuring changes in the refractive index near an organic functional film attached to the metal film of a chip by measuring a peak shift in the wavelength of reflected light, or changes in amounts of reflected light in a certain wavelength, so as to detect adsorption and desorption occurring near the surface. The QCM measurement technique is a technique of detecting adsorbed or desorbed mass at the ng level, using a change in frequency of a crystal due to adsorption or desorption of a substance on gold electrodes of a quartz crystal (device). In addition, the ultra-fine particle surface (nm level) of gold is functionalized, and physiologically active substances are immobilized thereon. Thus, a reaction to recognize specificity among physiologically active substances is carried out, thereby detecting a substance associated with a living organism from sedimentation of gold fine particles or sequences.

In all of the above-described techniques, the surface where a physiologically active substance is immobilized is important. Surface plasmon resonance (SPR), which is most commonly used in this technical field, will be described below as an example.

A commonly used measurement chip comprises a transparent substrate (e.g., glass), an evaporated metal film, and a thin film having thereon a functional group capable of immobilizing a physiologically active substance. The measurement chip immobilizes the physiologically active substance on the metal surface via the functional group. A specific binding reaction between the physiological active substance and a test substance is measured, so as to analyze an interaction between biomolecules.

As a thin film having a functional group capable of immobilizing a physiologically active substance, there has been reported a measurement chip where a physiologically active substance is immobilized by using a functional group binding to metal, a linker with a chain length of 10 or more atoms, and a compound having a functional group capable of binding to the physiologically active substance (Japanese Patent No. 2815120). Moreover, a measurement chip comprising a metal film and a plasma-polymerized film formed on the metal film has been reported (Japanese Patent Laid-Open No. 9-264843).

On the other hand, when a specific binding reaction is measured between a physiologically active substance and a test substance, the test substance does not necessarily consist of a single component, but it is sometimes required to measure the test substance existing in a heterogeneous system, such as in a cell extract. In such a case, if various contaminants such as proteins or lipids were non-specifically adsorbed on the detection surface, detection sensitivity in measurement would significantly be decreased. The aforementioned detection surface has been problematic in that such non-specific adsorption often takes place thereon.

A thin film formation method involving spin coating comprises adding a coating solution dropwise to a substrate to be coated and drawing the coating solution thereon by centrifugal force, so as to form a thin film. However, this method is problematic in that film thickness distribution is likely to occur. In order to solve such a problem, several methods have been studied. For example, a method comprising adding a coating solution dropwise to a substrate to be coated and then rotating the substrate in a hermetically sealed inner cup has been reported (Japanese Patent No. 2942213). As a modified method thereof, a method comprising spin coating while injecting thin gas into the inner cup has also been reported (Japanese Patent No. 3231970). However, these methods could not sufficiently prevent unevenness in the film thickness generated in the marginal part of the substrate. In particular, when a film is formed on a rectangular substrate by spin coating, such a substrate is disposed at a position that is deviated from the center of an inner cup, a coating solution is added dropwise thereto, and the substrate is then rotated, so as to form a film. However, in such a case also, a difference in film thickness distribution occurs.

### DISCLOSURE OF INVENTION

It is an object of the present invention to solve the aforementioned problems of the prior art techniques. In other words, it is an object of the present invention to provide a method for producing a solid substrate used for sensors having a film with a small film thickness distribution, and a solid surface used for sensors having a film with a small film thickness distribution. In particular, it is an object of the present invention to provide a method for producing a solid substrate used for sensors, the surface of which is coated with a thin polymer film with a small film thickness distribution, and a solid substrate used for sensors, the surface of which is coated with a thin polymer film with a small film thickness distribution.

As a result of intensive studies directed towards achieving the aforementioned objects, the present inventors have found that a thin film with a small film thickness distribution can be formed by rotating a substrate in a state where a substrate surface to be coated is inclined against the distal direction during coating, thereby completing the present invention. In addition, the present inventors have demonstrated that when a solid substrate used for sensors that is coated with a thin film formed by the above method is used, deviation in sensitivity due to unevenness in the film thickness can be suppressed.

Thus, the present invention provides a method for spin coating wherein the substrate is rotated in a state where the substrate surface to be coated is inclined against the rotation surface during coating.

Preferably, the substrate surface to be coated is not set on the rotation center of an inner cup, so that the distribution of centrifugal force acting on a coating solution on the surface can be reduced.

Preferably, the present invention is characterized in that the substrate surface to be coated is inclined outward or inward in the distal direction.

Preferably, such inclination of the substrate surface to be coated is between 5 degrees and 90 degrees.

In another aspect, the present invention provides a solid substrate used for sensors, on the surface of which a film is formed, which is produced by the aforementioned method of the present invention.

Preferably, the film is a hydrophobic polymer layer, which has a surface modification layer as the outermost layer from the substrate.

Preferably, the above-described surface modification layer has a functional group capable of generating a covalent bond.

Preferably, the solid substrate used for sensors of the present invention has a metal layer between the solid substrate and the hydrophobic polymer layer.

Preferably, the metal layer consists of a free-electron metal selected from the group consisting of gold, silver, copper, platinum, and aluminum.

Preferably, the solid substrate used for sensors of the present invention has a functional group capable of immobilizing a physiologically active substance on the outermost surface of the substrate.

Preferably, the functional group capable of immobilizing a physiologically active substance is —OH, —SH, —COOH, —NR¹R² (wherein each of R¹ and R² independently represents a hydrogen atom or a lower alkyl group), —CHO, —NR³NR¹R² (wherein each of R¹, R², and R³ independently represents a hydrogen atom or a lower alkyl group), —NCO, —NCS, an epoxy group, or a vinyl group.

Preferably, the solid substrate used for sensors of the present invention is used for non-electrochemical detection. More preferably, it is used for surface plasmon resonance analysis.

Preferably, a physiologically active substance is bound to the surface of the solid substrate used for sensors of the present invention.

In another aspect, the present invention provides a method for detecting or measuring a substance interacting with a physiologically active substance, which comprises: a step of allowing a physiologically active substance to come into contact with the surface of the aforementioned solid substrate used for sensors of the present invention, so as to immobilize it thereon; and a step of allowing the obtained solid substrate used for sensors, to the surface of which the physiologically active substance is bound, to come into contact with a test substance.

### BEST MODE FOR CARRYING OUT THE INVENTION

The embodiments of the present invention will be described below.

The spin coating application method of the present invention is characterized in that the substrate is rotated in a state where the substrate surface to be coated is inclined against the rotation surface during coating. By this technique, deviation in a coating solution on the substrate during spin coating is corrected, and the uniformity of a thin film formed on the coated surface is thereby improved.

As an embodiment of rotating a substrate in a state where the substrate surface to be coated is inclined against the rotation surface during coating, there are two cases: a case where rotation is carried out in a state where the substrate surface to be coated is inclined outward or inward in the centrifugal force direction of rotation; and the other case where rotation is carried out in a state where the substrate surface to be coated is inclined forward or backward in the rotation direction. Moreover, applying the aforementioned techniques in combination, the substrate can be rotated in a state where the substrate surface to be coated is inclined outward or inward in the centrifugal force direction of rotation, and at the same time, the substrate surface to be coated is inclined forward or backward in the rotation direction.

Preferably, a substrate can be disposed such that the surface to be coated does not exist on the rotation center of an inner cup. By doing so, the distribution of centrifugal force acting on a coating solution on the surface of the substrate can be reduced, thereby making film thickness distribution more uniform.

The angle of inclination of the substrate during spin coating can be determined as appropriate depending on the size of the substrate and the position of the substrate on the inner cup. It is preferably between 5 degrees and 90 degrees.

In the present invention, the substrate surface to be coated, to which a coating solution is added dropwise, is not particularly limited. It may be an upper surface, a lateral surface, or a lower surface.

In a preferred embodiment of the present invention, a substrate surface to be coated can be rotated in a state where generation of the gas flow towards the substrate is suppressed. The expression “generation of the gas flow towards the substrate is suppressed” is preferably used to mean that generation of the gas flow towards the substrate is suppressed to such an extent that a coating solution on the substrate surface to be coated is not dispersed to a direction opposite to the rotation direction due to wind pressure. A specific example of such a method of suppressing generation of the gas flow towards the substrate may include a method of enclosing the entire or a part of the periphery of the substrate to be coated with a vessel or a wall having a lateral surface other than the rotation center circle of an inner cup. More specific examples may include: a method of covering the front in the rotation direction of the substrate to be coated, both the front and the back thereof, or the entire lateral surfaces, with a windbreak board or a windbreak wall; and a method of disposing the substrate to be coated in a hermetically sealed vessel such as a box-type or capsule-type vessel. Otherwise, as an alternative method, it may also be possible to apply a method of adding a coating solution dropwise to the substrate surface to be coated and then rotating the substrate in a vacuum or under a reduced pressure.

The spin coating application method of the present invention is particularly advantageous when a film with a uniform film thickness is to be formed on a substrate having a surface to be coated, other than a perfect circle type. Moreover, when a coating solution is applied by spin coating to a surface to be coated, the form of which is an ellipse having a major axis that is 1.5 times or more longer than a minor axis, or a square, and particularly, a square having a long side that is 1.5 times or more longer than a short side, the spin coating application method of the present invention enables the formation of a thin polymer film with a small film thickness distribution on the surface.

A coating solution used in the present invention is preferably a hydrophobic polymer solution. A hydrophobic polymer that can be used in the present invention is substantially insoluble in water. Specifically, such a hydrophobic polymer has solubility in water of less than 0.1%. A hydrophobic polymer preferably contains 30% to 100% by weight of monomer having solubility in water at 25° C. of 0% to 20% by weight.

A hydrophobic monomer which forms a hydrophobic polymer can be selected from vinyl esters, acrylic esters, methacrylic esters, olefins, styrenes, crotonic esters, itaconic diesters, maleic diesters, fumaric diesters, allyl compounds, vinyl ethers, vinyl ketones, or the like. The hydrophobic polymer may be either a homopolymer consisting of one type of monomer, or copolymer consisting of two or more types of monomers.

Examples of a hydrophobic polymer that is preferably used in the present invention may include polystyrene, polyethylene, polypropylene, polyethylene terephthalate, polyvinyl chloride, polymethyl methacrylate, polyester, and nylon.

A hydrophobic polymer used in the present invention preferably has low water content. A preferred range of such water content is between 0.0001% and 0.1%. Specifically, water content is measured in accordance with the method described in ISO62. A hydrophobic polymer sheet with a square of 60 mm and a thickness of 1 mm is produced by the cast method, and the weight (W1) is then measured. Thereafter, this sheet is immersed in distilled water at 23° C. for 24 hours. After completion of the immersion, water on the surface of the sheet is wiped off, and the weight (W2) is then measured. Water content (%) is defined as (W2−W1)/W1×100.

The thickness of the hydrophobic polymer layer is not particularly limited. The total thickness of all the laminated polymer layers is preferably between 0.1 nm and 500 nm, and particularly preferably between 1 nm and 300 nm.

The substrate of the present invention preferably has a functional group capable of immobilizing a physiologically active substance on the outermost surface of the substrate. The term “the outermost surface of the substrate” is used to mean “the surface, which is farthest from the substrate,” and more specifically, it means “the surface of a hydrophobic polymer applied on a substrate, which is farthest from the substrate.”

In the present invention, the surface of the solid substrate is modified. Such a surface modification method can be selected, as appropriate, from chemical treatments using chemical agents, coupling agents, surfactants, or surface evaporation, and physical treatments using heating, ultraviolet rays, radioactive rays, plasma, or ions.

It is preferable that a functional group capable of generating a covalent bond as a result of surface modification be introduced. Preferred functional group includes —OH, —SH, —COOH, —NR¹R² (wherein each of R¹ and R² independently represents a hydrogen atom or lower alkyl group), —CHO, —NR³NR¹R² (wherein each of R¹, R² and R³ independently represents a hydrogen atom or lower alkyl group), —NCO, —NCS, an epoxy group, or a vinyl group. The number of carbon atoms contained in the lower alkyl group is not particularly limited herein. However, it is generally about C1 to C10, and preferably C1 to C6.

In order to introduce these functional groups into the surface, a method is applied that involves applying a hydrophobic polymer containing a precursor of such a functional group on a metal surface or metal film, and then generating the functional group from the precursor located on the outermost surface by chemical treatment. For example, polymethyl methacrylate, a hydrophobic polymer containing —COOCH₃ group, is applied on a metal film, and then the surface comes into contact with an NaOH aqueous solution (IN) at 40° C. for 16 hours, so that a —COOH group is generated on the outermost surface. In addition, when a polystyrene coating layer is subjected to a UV/ozone treatment for example, a —COOH group and a —OH group are generated on the outermost surface thereof.

A solid substrate used in the present invention is interpreted in the broadest sense. It indicates a base for supporting materials having functions. It includes not only hard materials, but also flexible materials such as a film or a sheet.

The solid substrate used in the present invention is preferably a metal surface or metal film, which is coated with a hydrophobic polymer. A metal constituting the metal surface or metal film is not particularly limited, as long as surface plasmon resonance is generated when the metal is used for a surface plasmon resonance biosensor. Examples of a preferred metal may include free-electron metals such as gold, silver, copper, aluminum or platinum. Of these, gold is particularly preferable. These metals can be used singly or in combination. Moreover, considering adherability to the above substrate, an interstitial layer consisting of chrome or the like may be provided between the substrate and a metal layer.

The film thickness of a metal film is not limited. When the metal film is used for a surface plasmon resonance biosensor, the thickness is preferably between 0.1 nm and 500 nm, and particularly preferably between 1 nm and 200 nm. If the thickness exceeds 500 nm, the surface plasmon phenomenon of a medium cannot be sufficiently detected. Moreover, when an interstitial layer consisting of chrome or the like is provided, the thickness of the interstitial layer is preferably between 0.1 nm and 10 nm.

Formation of a metal film may be carried out by common methods, and examples of such a method may include sputtering method, evaporation method, ion plating method, electroplating method, and nonelectrolytic plating method.

A metal film is preferably placed on a substrate. The description “placed on a substrate” is used herein to mean a case where a metal film is placed on a substrate such that it directly comes into contact with the substrate, as well as a case where a metal film is placed via another layer without directly coming into contact with the substrate. When a substrate used in the present invention is used for a surface plasmon resonance biosensor, examples of such a substrate may include, generally, optical glasses such as BK7, and synthetic resins. More specifically, materials transparent to laser beams, such as polymethyl methacrylate, polyethylene terephthalate, polycarbonate or a cycloolefin polymer, can be used. For such a substrate, materials that are not anisotropic with regard to polarized light and having excellent workability are preferably used.

The solid substrate of the present invention has as broad a meaning as possible, and means a sensor which converts an interaction between biomolecules into a signal such as an electric signal, so as to measure or detect a target substance. The solid substrate used for sensor according to the present invention can be used as a biosensor. The conventional biosensor is comprised of a receptor site for recognizing a chemical substance as a detection target and a transducer site for converting a physical change or chemical change generated at the site into an electric signal. In a living body, there exist substances having an affinity with each other, such as enzyme/substrate, enzyme/coenzyme, antigen/antibody, or hormone/receptor. The biosensor operates on the principle that a substance having an affinity with another substance, as described above, is immobilized on a substrate to be used as a molecule-recognizing substance, so that the corresponding substance can be selectively measured.

A physiologically active substance is covalently bound to the above-obtained solid substrate for sensor via the above functional group, so that the physiologically active substance can be immobilized on the metal surface or metal film.

A physiologically active substance immobilized on the substrate for sensor of the present invention is not particularly limited, as long as it interacts with a measurement target. Examples of such a substance may include an immune protein, an enzyme, a microorganism, nucleic acid, a low molecular weight organic compound, a nonimmune protein, an immunoglobulin-binding protein, a sugar-binding protein, a sugar chain recognizing sugar, fatty acid or fatty acid ester, and polypeptide or oligopeptide having a ligand-binding ability.

Examples of an immune protein may include an antibody whose antigen is a measurement target, and a hapten. Examples of such an antibody may include various immunoglobulins such as IgG, IgM, IgA, IgE or IgD. More specifically, when a measurement target is human serum albumin, an anti-human serum albumin antibody can be used as an antibody. When an antigen is an agricultural chemical, pesticide, methicillin-resistant Staphylococcus aureus, antibiotic, narcotic drug, cocaine, heroin, crack or the like, there can be used, for example, an anti-atrazine antibody, anti-kanamycin antibody, anti-metamphetamine antibody, or antibodies against O antigens 26, 86, 55, 111 and 157 among enteropathogenic Escherichia coli.

An enzyme used as a physiologically active substance herein is not particularly limited, as long as it exhibits an activity to a measurement target or substance metabolized from the measurement target. Various enzymes such as oxidoreductase, hydrolase, isomerase, lyase or synthetase can be used. More specifically, when a measurement target is glucose, glucose oxidase is used, and when a measurement target is cholesterol, cholesterol oxidase is used. Moreover, when a measurement target is an agricultural chemical, pesticide, methicillin-resistant Staphylococcus aureus, antibiotic, narcotic drug, cocaine, heroin, crack or the like, enzymes such as acetylcholine esterase, catecholamine esterase, noradrenalin esterase or dopamine esterase, which show a specific reaction with a substance metabolized from the above measurement target, can be used.

A microorganism used as a physiologically active substance herein is not particularly limited, and various microorganisms such as Escherichia coli can be used.

As nucleic acid, those complementarily hybridizing with nucleic acid as a measurement target can be used. Either DNA (including cDNA) or RNA can be used as nucleic acid. The type of DNA is not particularly limited, and any of native DNA, recombinant DNA produced by gene recombination and chemically synthesized DNA may be used.

As a low molecular weight organic compound, any given compound that can be synthesized by a common method of synthesizing an organic compound can be used.

A nonimmune protein used herein is not particularly limited, and examples of such a nonimmune protein may include avidin (streptoavidin), biotin, and a receptor.

Examples of an immunoglobulin-binding protein used herein may include protein A, protein G, and a rheumatoid factor (RF).

As a sugar-binding protein, for example, lectin is used.

Examples of fatty acid or fatty acid ester may include stearic acid, arachidic acid, behenic acid, ethyl stearate, ethyl arachidate, and ethyl behenate.

A biosensor to which a physiologically active substance is immobilized as described above can be used to detect and/or measure a substance which interacts with the physiologically active substance.

In the present invention, it is preferable to detect and/or measure an interaction between a physiologically active substance immobilized on the solid substrate for sensor and a test substance by a nonelectric chemical method. Examples of a non-electrochemical method may include a surface plasmon resonance (SPR) measurement technique, a quartz crystal microbalance (QCM) measurement technique, and a measurement technique that uses functional surfaces ranging from gold colloid particles to ultra-fine particles.

In a preferred embodiment of the present invention, the biosensor of the present invention can be used as a biosensor for surface plasmon resonance which is characterized in that it comprises a metal film placed on a transparent substrate.

A biosensor for surface plasmon resonance is a biosensor used for a surface plasmon resonance biosensor, meaning a member comprising a portion for transmitting and reflecting light emitted from the sensor and a portion for immobilizing a physiologically active substance. It may be fixed to the main body of the sensor or may be detachable.

The surface plasmon resonance phenomenon occurs due to the fact that the intensity of monochromatic light reflected from the border between an optically transparent substance such as glass and a metal thin film layer depends on the refractive index of a sample located on the outgoing side of the metal. Accordingly, the sample can be analyzed by measuring the intensity of reflected monochromatic light.

A device using a system known as the Kretschmann configuration is an example of a surface plasmon measurement device for analyzing the properties of a substance to be measured using a phenomenon whereby a surface plasmon is excited with a lightwave (for example, Japanese Patent Laid-Open No. 6-167443). The surface plasmon measurement device using the above system basically comprises a dielectric block formed in a prism state, a metal film that is formed on a face of the dielectric block and comes into contact with a measured substance such as a sample solution, a light source for generating a light beam, an optical system for allowing the above light beam to enter the dielectric block at various angles so that total reflection conditions can be obtained at the interface between the dielectric block and the metal film, and a light-detecting means for detecting the state of surface plasmon resonance, that is, the state of attenuated total reflection, by measuring the intensity of the light beam totally reflected at the above interface.

In order to achieve various incident angles as described above, a relatively thin light beam may be caused to enter the above interface while changing an incident angle. Otherwise, a relatively thick light beam may be caused to enter the above interface in a state of convergent light or divergent light, so that the light beam contains components that have entered therein at various angles. In the former case, the light beam whose reflection angle changes depending on the change of the incident angle of the entered light beam can be detected with a small photodetector moving in synchronization with the change of the above reflection angle, or it can also be detected with an area sensor extending along the direction in which the reflection angle is changed. In the latter case, the light beam can be detected with an area sensor extending to a direction capable of receiving all the light beams reflected at various reflection angles.

With regard to a surface plasmon measurement device with the above structure, if a light beam is allowed to enter the metal film at a specific incident angle greater than or equal to a total reflection angle, then an evanescent wave having an electric distribution appears in a measured substance that is in contact with the metal film, and a surface plasmon is excited by this evanescent wave at the interface between the metal film and the measured substance. When the wave vector of the evanescent light is the same as that of a surface plasmon and thus their wave numbers match, they are in a resonance state, and light energy transfers to the surface plasmon. Accordingly, the intensity of totally reflected light is sharply decreased at the interface between the dielectric block and the metal film. This decrease in light intensity is generally detected as a dark line by the above light-detecting means. The above resonance takes place only when the incident beam is p-polarized light. Accordingly, it is necessary to set the light beam in advance such that it enters as p-polarized light.

If the wave number of a surface plasmon is determined from an incident angle causing the attenuated total reflection (ATR), that is, an attenuated total reflection angle (θSP), the dielectric constant of a measured substance can be determined. As described in Japanese Patent Laid-Open No. 11-326194, a light-detecting means in the form of an array is considered to be used for the above type of surface plasmon measurement device in order to measure the attenuated total reflection angle (θSP) with high precision and in a large dynamic range. This light-detecting means comprises multiple photo acceptance units that are arranged in a certain direction, that is, a direction in which different photo acceptance units receive the components of light beams that are totally reflected at various reflection angles at the above interface.

In the above case, there is established a differentiating means for differentiating a photodetection signal outputted from each photo acceptance unit in the above array-form light-detecting means with regard to the direction in which the photo acceptance unit is arranged. An attenuated total reflection angle (OSP) is then specified based on the derivative value outputted from the differentiating means, so that properties associated with the refractive index of a measured substance are determined in many cases.

In addition, a leaking mode measurement device described in “Bunko Kenkyu (Spectral Studies)” Vol. 47, No. 1 (1998), pp. 21 to 23 and 26 to 27 has also been known as an example of measurement devices similar to the above-described device using attenuated total reflection (ATR). This leaking mode measurement device basically comprises a dielectric block formed in a prism state, a clad layer that is formed on a face of the dielectric block, a light wave guide layer that is formed on the clad layer and comes into contact with a sample solution, a light source for generating a light beam, an optical system for allowing the above light beam to enter the dielectric block at various angles so that total reflection conditions can be obtained at the interface between the dielectric block and the clad layer, and a light-detecting means for detecting the excitation state of waveguide mode, that is, the state of attenuated total reflection, by measuring the intensity of the light beam totally reflected at the above interface.

In the leaking mode measurement device with the above structure, if a light beam is caused to enter the clad layer via the dielectric block at an incident angle greater than or equal to a total reflection angle, only light having a specific wave number that has entered at a specific incident angle is transmitted in a waveguide mode into the light wave guide layer, after the light beam has penetrated the clad layer. Thus, when the waveguide mode is excited, almost all forms of incident light are taken into the light wave guide layer, and thereby the state of attenuated total reflection occurs, in which the intensity of the totally reflected light is sharply decreased at the above interface. Since the wave number of a waveguide light depends on the refractive index of a measured substance placed on the light wave guide layer, the refractive index of the measurement substance or the properties of the measured substance associated therewith can be analyzed by determining the above specific incident angle causing the attenuated total reflection.

In this leaking mode measurement device also, the above-described array-form light-detecting means can be used to detect the position of a dark line generated in a reflected light due to attenuated total reflection. In addition, the above-described differentiating means can also be applied in combination with the above means.

The above-described surface plasmon measurement device or leaking mode measurement device may be used in random screening to discover a specific substance binding to a desired sensing substance in the field of research for development of new drugs or the like. In this case, a sensing substance is immobilized as the above-described measured substance on the above thin film layer (which is a metal film in the case of a surface plasmon measurement device, and is a clad layer and a light guide wave layer in the case of a leaking mode measurement device), and a sample solution obtained by dissolving various types of test substance in a solvent is added to the sensing substance. Thereafter, the above-described attenuated total reflection angle (OSP) is measured periodically when a certain period of time has elapsed.

If the test substance contained in the sample solution is bound to the sensing substance, the refractive index of the sensing substance is changed by this binding over time. Accordingly, the above attenuated total reflection angle (OSP) is measured periodically after the elapse of a certain time, and it is determined whether or not a change has occurred in the above attenuated total reflection angle (OSP), so that a binding state between the test substance and the sensing substance is measured. Based on the results, it can be determined whether or not the test substance is a specific substance binding to the sensing substance. Examples of such a combination between a specific substance and a sensing substance may include an antigen and an antibody, and an antibody and an antibody. More specifically, a rabbit anti-human IgG antibody is immobilized as a sensing substance on the surface of a thin film layer, and a human IgG antibody is used as a specific substance.

It is to be noted that in order to measure a binding state between a test substance and a sensing substance, it is not always necessary to detect the angle itself of an attenuated total reflection angle (OSP). For example, a sample solution may be added to a sensing substance, and the amount of an attenuated total reflection angle (OSP) changed thereby may be measured, so that the binding state can be measured based on the magnitude by which the angle has changed. When the above-described array-form light-detecting means and differentiating means are applied to a measurement device using attenuated total reflection, the amount by which a derivative value has changed reflects the amount by which the attenuated total reflection angle (OSP) has changed. Accordingly, based on the amount by which the derivative value has changed, a binding state between a sensing substance and a test substance can be measured (Japanese Patent Application No. 2000-398309 filed by the present applicant). In a measuring method and a measurement device using such attenuated total reflection, a sample solution consisting of a solvent and a test substance is added dropwise to a cup- or petri dish-shaped measurement chip wherein a sensing substance is immobilized on a thin film layer previously formed at the bottom, and then, the above-described amount by which an attenuated total reflection angle (OSP) has changed is measured.

Moreover, Japanese Patent Laid-Open No. 2001-330560 describes a measurement device using attenuated total reflection, which involves successively measuring multiple measurement chips mounted on a turntable or the like, so as to measure many samples in a short time.

When the biosensor of the present invention is used in surface plasmon resonance analysis, it can be applied as a part of various surface plasmon measurement devices described above.

The present invention will be further specifically described in the following examples. However, the examples are not intended to limit the scope of the present invention.

### EXAMPLES

### Example 1

### Angle-fixed Inclination Spin Coating

(1) Preparation of polymethyl methacrylate-polystyrene Copolymer (Hereinafter Abbreviated as PMMA/PSt) Solution

3.0 g of PMMA/PSt (number average molecular weight: 6,000) was dissolved in 2-acetoxy-1-methoxypropane. Thereafter, 2-acetoxy-1-methoxypropane was added thereto, resulting in a liquid amount of 100 ml, so as to prepare 3.0% PMMA/PSt.

(2) Film Formation by Spin Coating

A glass substrate with a size of 8 mm long×80 mm wide×0.5 mm thick, which had been coated with gold via evaporation resulting in a gold film with a thickness of 50 nm, was treated with a Model-208 UV-ozone cleaning system (TECHNOVISION INC.) for 30 minutes. Thereafter, this glass substrate was placed in an aluminum vessel having a hermetically sealed structure of 40 mm long×120 mm wide×20 mm deep. Thereafter, this aluminum vessel was fixed on the inner cup of a spin-coater equipped with a hermetically sealed inner cup (MODEL SC408 (patented); manufactured by Nanotec Corp.) at a position wherein the glass substrate was disposed at a position that was 135 mm from the center, in a direction wherein the tangential view of a circular arc became a long axis, and in a state where the vessel was inclined at 30 degrees in the rotation center direction against the surface of the inner cup. Thereafter, using a micropipette, 200 μl of 3.0% PMMA/PSt was added dropwise to the glass substrate, so that the entire surface of the glass substrate was coated with 3.0% PMMA/Pst. The aluminum vessel was hermetically sealed and then rotated at 200 rpm. 60 seconds later, the rotation was terminated. A spin coating chip was removed from the aluminum vessel, and it was then dried overnight at 60° C. under ordinary pressure. The film thickness distribution of a width of 5 mm in the central part of the substrate was measured at intervals of 0.1 mm in the longitudinal direction (short side) by the ellipsometry method (In-Situ Ellipsometer MAUS-101; manufactured by Five Lab). As a result, the mean film thickness was found to be 20 nm, and the coefficient of variation of the film thickness (the percentage of the value obtained by dividing the standard deviation by the mean value) was found to be 10%.

### Example 2

### Angle-Variable Inclination Spin Coating

(1) Preparation of polymethyl methacrylate-polystyrene Copolymer (Hereinafter Abbreviated as PMMA/PSt) Solution (2)

0.5 g of PMMA/PSt (number average molecular weight: 6,000) was dissolved in 2-acetoxy-1-methoxypropane. Thereafter, 2-acetoxy-1-methoxypropane was added thereto, resulting in a liquid amount of 100 ml, so as to prepare 0.5% PMMA/PSt.

(2) Film Formation by Spin Coating

The same glass substrate as used in Example 1 was placed in an aluminum vessel having a hermetically sealed structure of 40 mm long×120 mm wide×20 mm deep. Thereafter, a swing-type table that was movable in the centrifugal force direction was established at a position that was 100 mm from the center of the inner cup of a spin-coater equipped with a hermetically sealed inner cup (MODEL SC408 (patented); manufactured by Nanotec Corp.), and the above aluminum vessel was then fixed on the swing table. Subsequently, using a micropipette, 200 μl of 0.5% PMMA/PSt was added dropwise to the glass substrate, so that the entire surface of the glass substrate was coated with 0.5% PMMA/Pst. The aluminum vessel was hermetically sealed and then rotated at 200 rpm. 60 seconds later, rotation was terminated. During spin coating, the achieved inclination angle of the aluminum vessel was about 80 degrees in the centrifugal force direction against the surface of the inner cup. A spin coating chip was removed from the aluminum vessel, and it was then dried overnight at 60° C. under ordinary pressure. The film thickness distribution of a width of 5 mm in the central part of the substrate was measured at intervals of 0.1 mm in the longitudinal direction (short side) by the ellipsometry method (In-Situ Ellipsometer MAUS-101; manufactured by Five Lab). As a result, the mean film thickness was found to be 20 nm, and the coefficient of variation of the film thickness (the percentage of the value obtained by dividing the standard deviation by the mean value) was found to be 6%.

### Comparative Example 1

### Angle-Fixed Horizontal Spin Coating

The same operations as in Example 1 were carried out with the exception that the aluminum vessel containing the glass substrate was horizontally placed on the inner cup in Example 1(2). As a result, the mean film thickness was found to be 20 nm, and the coefficient of variation of the film thickness (the percentage of the value obtained by dividing the standard deviation by the mean value) was found to be 35%.

### Test Example 1: Mouse IgG-Binding Experiment

(1) Introduction of COOH Group into PMMA/PSt Surface

The spin coating chip produced in each of Example 1, Example 2, and Comparative Example 1, was immersed in an aqueous NaOH solution (1 N) at 60° C. for 16 hours. Thereafter, the chip was washed with water 3 times. The obtained sample is called a COOH-modified spin coating chip.

(2) Immobilization of Protein A

A mixed solution consisting of 1-ethyl-2,3-dimethylaminopropylcarbodiimide (200 mM) and N-hydroxysuccinimide (50 mM) was allowed to come into contact with the spin coating chip produced by the aforementioned method for 30 minutes. Subsequently, the chip was washed with a 50 mM acetate buffer (pH 4.5; manufactured by Biacore). Thereafter, a protein A (manufactured by Nacalai Tesque) solution (100 μg/ml, 50 mM acetate buffer, pH 4.5) was allowed to come into contact with the chip for 30 minutes. The chip was then washed with a 50 mM acetate buffer (pH 4.5).

Moreover, an ethanolamine-HCl solution (1 M, pH 8.5) was allowed to come into contact with the chip for 30 minutes. Thereafter, the chip was washed with a 50 mM acetate buffer (pH 4.5), so as to block activated COOH groups, which had not been reacted with protein A and remained.

Furthermore, an aqueous NaOH solution (10 mM) was allowed to come into contact with the chip for 1 minute. Thereafter, the chip was washed with an HBS-EP buffer (0.01 mol/l HEPES (N-2-hydroxyethylpiperazine-N′-2-ethanesulfonic acid) (pH 7.4), 0.15 mol/l NaCl, 0.003 mol/l EDTA, and 0.005% by weight of Surfactant P20; manufactured by Biacore), so as to eliminate protein A, which had non-specifically adsorbed on the surface of the spin coating chip. The obtained sample is called a protein A-immobilized chip.

(3) Detection of Binding Signals of Mouse IgG

Regardless of the detected position of the protein A-immobilized chip, the more stable binding signals, the higher reliability that can be obtained from the experiment. The dependence of the binding amount of mouse IgG on the position of the protein A-immobilized chip was evaluated by the following method.

The protein A-immobilized chip produced by the steps described in (2) above was placed in a surface plasmon resonance device (the SPR device shown in FIG. 5 of Applied Spectroscopy, 42(8), 1375-1379 (1988)). The chip was set in the above device, such that the center position, to which laser beam was applied, became the center in the horizontal direction (long side axis), and such that it became the center and the position that was 1 mm apart from the center in the longitudinal direction (short side). A polypropylene material was placed on the chip, so as to produce a cell with a width (longitudinal direction) of 5 mm, a length (horizontal direction) of 7.5 mm, and a depth of 1 mm.

The inside of the measurement cell was filled with an HBS-EP buffer, and measurement was then initiated. The inside of the cell was replaced with a mouse IgG (manufactured by Cosmo Bio) solution (10 μg/ml, HBS-EP buffer), and it was then left at rest for 5 minutes. Signal change was calculated after 5 minutes.

Thereafter, the inside of the cell was allowed to come into contact with an aqueous NaOH solution (10 mM) for 1 minute, and it was then washed with an HBS-EP buffer. It was confirmed that the binding of mouse IgG was disconnected by the above operation and that the signal was returned to the baseline.

The chip was fixed at a position that was further 10 mm apart from the end in the horizontal direction, and the binding of mouse IgG was measured in the same manner. Thereafter, the chips were placed at intervals of 10 mm from the end in the horizontal direction, and thus, measurement was carried out at 7 points for this protein A-immobilized chip.

(4) Results

The signal change caused by the binding of mouse IgG that depends on the position in the chip is shown in Table 1. The term “CV” represents the coefficient of variation (the percentage of the value obtained by dividing the standard deviation by the mean value).

TABLE 1

| | Distance in longitudinal direction | | | | |
|---|---|---|---|---|---|
| | Signal change (RU) | | | | |
| Sample | | | | Mean | |
| Name | −1 mm | Center | +1 mm | value | CV (%) |
| Example 1 | 620 | 610 | 430 | 550 | 20 |
| Example 2 | 620 | 620 | 490 | 580 | 13 |
| Comparative | 630 | 610 | 300 | 510 | 36 |
| example 1 | | | | | |

(With regard to the distance in the longitudinal direction, the rotation inside during spin coating is represented by the symbol - (minus), and the rotation outside is represented by the symbol +(plus).)

From the results shown in Table 1, it is found that when the spin coating chip of the present invention is used, the film thickness distribution of the PMMA/PSt film was small in the chip, and that deviation in signal change due to position is also small.

Effect of the Invention

The method of the present invention enables formation of a thin film with a small film thickness distribution on a substrate. That is, the present invention provides a solid substrate used for sensors having a film with a small film thickness distribution, and particularly, a solid substrate used for sensors, the surface of which is coated with a thin polymer film. Using a solid substrate used for sensors produced by the method of the present invention, it becomes possible to conduct measurement with suppressed deviation in sensor detection sensitivity.

1. A method for spin coating wherein the substrate is rotated in a state where the substrate surface to be coated is inclined against the rotation surface during coating.

2. The method for spin coating according to claim 1 wherein the rotation is carried out in a state where the substrate surface to be coated is inclined outward or inward in the centrifugal force direction of rotation.

3. The method for spin coating according to claim 1 wherein the rotation is carried out in a state where the substrate surface to be coated is inclined forward or backward in the rotation direction.

4. The method for spin coating according to claim 1 wherein the substrate surface to be coated is not set on the rotation center of an inner cup.

5. The method for spin coating according to claim 1 wherein the inclination of the substrate surface to be coated is between 5 degrees and 90 degrees.

6. A solid substrate used for sensors, on the surface of which a film is formed, which is produced by the method of claim 1.

7. The solid substrate used for sensors according to claim 6 wherein the film is a hydrophobic polymer layer, and which has a surface modification layer as the outermost layer from the substrate.

8. The solid substrate used for sensors according to claim 7 wherein the surface modification layer has a functional group capable of generating a covalent bond.

9. The solid substrate used for sensors according to claim 7 which has a metal layer between the solid substrate and the hydrophobic polymer layer.

10. The solid substrate used for sensors according to claim 9 wherein the metal layer consists of a free-electron metal selected from the group consisting of gold, silver, copper, platinum, and aluminum.

11. The solid substrate used for sensors according to claim 6 which has a functional group capable of immobilizing a physiologically active substance on the outermost surface of the substrate.

12. The solid substrate used for sensors according to claim 11 wherein the functional group capable of immobilizing a physiologically active substance is —OH, —SH, —COOH, —NR¹R² (wherein each of R¹ and R² independently represents a hydrogen atom or a lower alkyl group), —CHO, —NR³NR¹R² (wherein each of R¹, R², and R³ independently represents a hydrogen atom or a lower alkyl group), —NCO, —NCS, an epoxy group, or a vinyl group.

13. The solid substrate used for sensors according to claim 6 which is used for non-electrochemical detection.

14. The solid substrate used for sensors according to claim 6 which is used for surface plasmon resonance analysis.

15. The solid substrate used for sensors according to claim 6, to the surface of which a physiologically active substance is bound.

16. A method for detecting or measuring a substance interacting with a physiologically active substance, which comprises: a step of allowing a physiologically active substance to come into contact with the surface of the solid substrate used for sensors according to claim 6, so as to immobilize it thereon; and a step of allowing the obtained solid substrate used for sensors, to the surface of which the physiologically active substance is bound, to come into contact with a test substance.
